(19) United States
(12) Patent Application Publication
(10) Pub. No.: US 2019/0029875 A1
(43) Pub. Date: Jan. 31, 2019

(54) PREDICTION OF BLEEDING PATTERNS IN WOMEN AFTER INSERTION OF AN INTRAUTERINE ACTIVE-INGREDIENT-RELEASING SYSTEM

(71) Applicant: BAYER AKTIENGESELLSCHAFT, Leverkusen (DE)

(72) Inventors: Ann-Kathrin FRENZ, Essen (DE); Christiane AHLERS, Hagen (DE); Vita BECKERT, Berlin (DE); Christoph GERLINGER, Berlin (DE)

(73) Assignee: BAYER AKTIENGESELLSCHAFT, Leverkusen (DE)

(21) Appl. No.: 16/048,068

(22) Filed: Jul. 27, 2018

(30) Foreign Application Priority Data
Jul. 28, 2017 (EP) 17183705.7
May 2, 2018 (EP) 18170360.4

(51) Int. Cl. A61F 6/18; A61B 10/00

(52) U.S. Cl. CPC A61F 6/18; A61B 5/4325; A61B 10/0012

(57) ABSTRACT

Provided are systems, methods, and computer programs for predicting the menstrual bleeding pattern of a woman using an intrauterine active-ingredient-releasing system. Some embodiments comprise acquiring data on the bleeding pattern of a woman during a period of time beginning with insertion of an intrauterine active-ingredient-releasing system and acquiring one or a plurality of predictor values. Some embodiments include determining a future bleeding pattern of the woman based a classification model configured to classify the woman in one of several bleeding pattern categories. Additionally, some embodiments may comprise displaying the determined future bleeding pattern to one or more of the woman or a physician.

### CROSS REFERENCE TO RELATED APPLICATIONS

This application claims the benefit of European Application No. 18170360.4, filed May 2, 2018 and European Application No. 17183705.7, filed Jul. 28, 2017.

### FIELD

The present invention concerns the prediction of menstrual bleeding patterns in women in whom an intrauterine active-ingredient-releasing system has been inserted. The subject matter of the present invention is a system, a method, and a computer program product for predicting menstrual bleeding patterns.

### BACKGROUND

An intrauterine active-ingredient-releasing system is an intrauterine device that comprises one or a plurality of active ingredients and is inserted into the uterus of a woman. After insertion, it slowly releases the active ingredient or plurality of active ingredients over a period of time. An example of an intrauterine active-ingredient-releasing system is a hormone-releasing IUD (intrauterine device) that slowly and steadily releases a progestin in the uterus, for example in order to prevent pregnancy and/or for the treatment of idiopathic menorrhagia or dysmenorrhea.

In the natural course of the cycle, the endometrium thickens, and if pregnancy does not occur, it is sloughed and discharged though the vagina via the cervical canal. This is perceived as menstrual bleeding. The extent of menstrual bleeding may vary depending on the extent of endometrial thickening.

In use of an intrauterine active-ingredient-releasing system, thickening of the endometrium is affected by the active ingredient released. For example, with the use of a progestin-containing hormone-releasing IUD, there is little endometrial thickening due to the progestogenic action. The menstrual flow becomes lighter, less painful, and can even be completely absent (cf. for example D. Socolov et al., The European Journal of Contraception and Reproductive Health Care, December 2011; 16: 480-487).

Accordingly, intrauterine active-ingredient-releasing systems can cause changes in bleeding patterns. In particular, unexpected bleeding may occur during the adaptation phase, constituting a negative experience for women who have previously had regular menstrual bleeding.

For this reason, many women are anxious and/or dissatisfied the first time they use an intrauterine active-ingredient-releasing system. Many women have the inserted intrauterine active-ingredient-releasing system prematurely removed—in some cases, however, menstrual bleeding in these women would have settled into a reliable pattern. Conversely, it may be the case in other women that lasting problems occur, making removal of the intrauterine active-ingredient-releasing system advisable.

There are solutions for predicting the menstrual cycle (US 2016/0196383 A1, US 2016/0100826 A1), in particular for determining fertile days (US 2010/0191696 A1, U.S. Pat. No. 5,836,890), but these cannot applied in women using an intrauterine active-ingredient-releasing system. E. T. de Jonge et al. conducted a study involving the identification of predictors for oligomenorrhea in women using an intrauterine active-ingredient-releasing system (Contraception 76 (2007) 91-95). However, the approach they used in this study is not applicable to prediction of general bleeding patterns. The purpose of the study was to identify factors prior to insertion of the intrauterine active-ingredient-releasing system that are associated with oligomenorrhea 12 months after insertion.

### BRIEF SUMMARY

Methods, systems, and computer programs are provided to predict bleeding patterns in women using an intrauterine active-ingredient-releasing system.

As described above, there are solutions for predicting the menstrual cycle (US 2016/0196383 A1, US 2016/0100826 A1), in particular for determining fertile days (US 2010/0191696 A1, U.S. Pat. No. 5,836,890), but these cannot applied in women using an intrauterine active-ingredient-releasing system. E. T. de Jonge et al. conducted a study involving the identification of predictors for oligomenorrhea in women using an intrauterine active-ingredient-releasing system (Contraception 76 (2007) 91-95). However, the approach they used in this study is not applicable to prediction of general bleeding patterns. The purpose of the study was to identify factors prior to insertion of the intrauterine active-ingredient-releasing system that are associated with oligomenorrhea 12 months after insertion. On the one hand, prediction for a period 12 months after insertion of the intrauterine active-ingredient-releasing system is relatively late from a temporal standpoint; for many women, it would be advantageous to obtain data on future bleeding patterns at an earlier point in time. On the other hand, and this is confirmed by our own studies, prediction of the bleeding pattern based solely on predictors determined prior to insertion of the intrauterine active-ingredient-releasing system has been found to be unsuccessful.

In some embodiments described herein, methods for predicting bleeding patterns in women using an intrauterine active-ingredient-releasing system comprise developing a prediction model. For example, in a study on bleeding patterns of women using an intrauterine active-ingredient-releasing system for a period 61-180 days after insertion of said intrauterine active-ingredient-releasing system, categories showing respectively similar bleeding patterns were identified in the data, and in a further step, it was investigated whether and how these categories can be predicted based on data collected before the aforementioned time period. This made it possible to develop a prediction model allowing prediction of the bleeding patterns of women based on predictor values determined during a prediction period subsequent to the observation period.

Moreover, a solution was found that allows the regularity of predicted bleeding to be defined and also predicted.

In some embodiments, a system for predicting the menstrual bleeding pattern of a woman using an intrauterine active-ingredient-releasing system is provided, the system comprising
an acquisition unit for acquiring predictor values, wherein the acquisition unit is configured such that it acquires data on a woman's bleeding pattern in an observation period, wherein the observation period begins with insertion of the intrauterine active-ingredient-releasing system and lasts at least 60 days,
prediction unit that is configured such that it determines the future bleeding pattern of the woman based on the predictor values, wherein the predictor unit is based on a classification model that classifies the woman in one of several bleeding pattern categories, wherein the bleeding pattern categories differ with respect to bleeding intensities to be expected in a prediction time period, wherein the prediction time period is subsequent to the observation period, and
an output unit that is configured such that it displays the determined future bleeding pattern to the woman and/or another person, in particular a physician.

In some embodiments, a method for predicting the bleeding pattern of a woman using an intrauterine active-ingredient-releasing system is provided, the method comprising:
acquisition of the bleeding pattern of the woman in an observation period, wherein the observation period begins with insertion of the intrauterine active-ingredient-releasing system and lasts at least 60 days, and optional acquisition of further predictor values,
determination of the future bleeding pattern of the woman based on the predictor values using a classification model wherein the classification model classifies the woman in one of several bleeding pattern categories, wherein the bleeding pattern categories differ with respect to bleeding intensities to be expected in a prediction time period, wherein the prediction time period is subsequent to the observation period, and
outputting of the determined future bleeding pattern to the woman and/or another person, in particular a physician.

In some embodiments, a computer program product comprising a data carrier on which a computer program can be stored in the working memory of one or a plurality of computer systems is provided, wherein the computer program product causes the computer system/systems to carry out the following steps:
acquisition of the bleeding pattern of a woman using an intrauterine active-ingredient-releasing system in an observation period, wherein the observation period begins with insertion of the intrauterine active-ingredient-releasing system and lasts at least 60 days, and optional acquisition of further predictor values,
determination of the future bleeding pattern of the woman based on the predictor values using a classification model, wherein said classification model classifies the woman in one of several bleeding pattern categories, wherein the bleeding pattern categories differ with respect to bleeding intensities to be expected in a prediction time period, wherein the prediction time period is subsequent to the observation period, and
outputting of the determined future bleeding pattern to the woman and/or another person, in particular a physician.

In the following, the invention is explained in further detail without distinguishing among the various embodiments (method, system, computer program product). Rather, the following description is to apply analogously to any and/or all embodiments of the invention, regardless of the context in which it is presented (method, system, computer program product).

The present invention allows prediction of the future bleeding pattern of a woman wearing/using an intrauterine active-ingredient-releasing system.

An “intrauterine active-ingredient-releasing system” is an intrauterine device that is inserted in the uterus of a woman, where it releases one or a plurality of active ingredients.

An “active ingredient” is a substance or a substance mixture that has a specific action and produces a specific reaction in an organism. In particular, an active ingredient within the meaning of some embodiments is a substance or a substance mixture that exerts an action on a woman's endometrium.

An example of an active ingredient is a hormone such as a progestogen.

“Progestogens” are steroids having a pregnane (10β,13β-dimethyl-17β-ethyl-gonane) backbone. The most important representatives are pregnanediol, progesterone and pregnenolone. In order to distinguish the natural progestogens from synthetic hormones, the latter are referred to as “progestins” or “synthetic progestogens.” An example of a progestogen is levonorgestrel, a synthetic second-generation progestin used for hormonal contraception (prevention of pregnancy). Levonorgestrel is also used in the hormone-releasing IUD Kyleena®.

In an embodiment, the intrauterine active-ingredient-releasing system is a hormone-releasing IUD (such as Mirena®, Kyleena® or Jaydess®), preferably a hormone-releasing IUD that comprises levonorgestrel as an active ingredient, such as the product available under the brand name Kyleena® (cf. for example https://www.patienteninfo-service.de/a-z-liste/k/kyleenaR-195-mg-intrauterine-wirkstofffreisetzungssystem/). A further example of a preferred intrauterine active-ingredient-releasing system is a system comprising indomethacin and levonorgestrel as active ingredients.

The term “bleeding pattern” is understood to indicate the occurrence or non-occurrence of menstrual bleeding in the woman. Examples of parameters that can characterise bleeding patterns include the intensity of bleeding (bleeding intensity), the duration of bleeding, and the regularity of bleeding. Indication of the bleeding intensity can be in the form of yes/no statement (bleeding/no bleeding) or in the form of a three-level classification (no bleeding/spotting/bleeding) or in the form of even more levels.

The term “prediction of the future bleeding pattern” is understood to refer to an indication as to the nature of the bleeding pattern in the future based on calculations. The indication therefore carries some uncertainty. The extent of the probability that the predicted pattern will actually occur can be determined and indicated.

The predictions made based on some embodiments meet the criteria for a valid prognosis: non-triviality, objectivity, and validity.

Prediction of the future bleeding pattern is carried out based on predictors.

A “predictor” is generally understood to be a variable that is used in order to predict or model the values of another variable. In the present case, predictors are used in order to predict the bleeding pattern of a woman using an intrauterine active-ingredient-releasing system.

A predictor according to embodiments described herein can be a (physically) measurable parameter such as the number of days during an observation period on which bleeding occurred. However, it can also be a subjective parameter such as the intensity of bleeding estimated by the woman herself.

As a rule, a predictor can comprise a plurality of values, also referred to in the following as predictor values. Values can be numerals together with a measuring unit, such as the age of a woman (e.g. 32 years). Values can also be numerals without a measuring unit, such as the number of pregnancies before insertion of an intrauterine active-ingredient-releasing system. Values can also be expressed in the form of words; for example, the self-estimated intensity of bleeding can be expressed with one of the following terms: “very light”, “light”, “heavy”, or “very heavy”.

Possible predictors for predicting the menstrual bleeding pattern are data on the woman using an intrauterine active-ingredient-releasing system, such as:
the age of the woman,
the contraception method the woman used prior to use of the intrauterine active-ingredient-releasing system,
the number of previous pregnancies/births of the woman,
the (regular) consumption of tablets and/or additive drugs (such as cigarettes),
information about the bleeding pattern in a defined time period (e.g. three months) before insertion of an intrauterine active-ingredient-releasing system.

Genetic data, data on food consumption, physical fitness, and other data on a woman's health status are also conceivable as predictors.

For example, predictors can be determined in studies or identified by analysing ongoing/completed studies.

Preferred predictors are data on the bleeding pattern during an observation period after the insertion of the intrauterine active-ingredient-releasing system, including:
points in time at which bleeding occurred during the observation period,
periods of time during which bleeding continued,
intensity of bleeding,
total number of bleeding days,
total number of spotting days,
regularity of bleeding (with respect to duration and intensity), and
number of bleeding or spotting episodes.

The observation period begins with the insertion of the intrauterine active-ingredient-releasing system and lasts at least 60 days. In some embodiments, the duration of the observation period is in the range of 61 days to 120 days. In some embodiments, the duration of the observation period is in the range of 70 days to 110 days, in the range of 80 to 100 days, in the range of 85 to 95 days, or in the range of 88 to 90 days.

It has been found that the bleeding pattern during an observation period beginning with insertion of the intrauterine active-ingredient-releasing system provides predictors that can be used to predict the future bleeding pattern, i.e. the bleeding pattern in a period of time following the observation period (prediction time period). According to some embodiments, a physician and/or the patient can determine at the latest after conclusion of the observation period the probable nature of the bleeding pattern in the future, so that the physician and the patient can discuss whether it is advisable to continue the treatment.

In some embodiments, the observation period therefore extends from the time of insertion of the intrauterine active-ingredient-releasing system until the time of a first, second or third follow-up examination, e.g. by a physician (a health professional, health practitioner, or health care provider). It is conceivable for a woman in whom the intrauterine active-ingredient-releasing system has been inserted to be asked to come in for a follow-up examination four weeks, five weeks, six weeks, seven weeks, eight weeks, nine weeks, 10 days, 20 days, 30 days, 40 days, 50 days, 60 days, 70 days, 80 days, 90 days, 100 days, 110 days, or 120 days or another time period after the insertion of the intrauterine active-ingredient-releasing system.

A computer is preferably used for the determination and/or collection of predictor values. It is also conceivable to use a plurality of computers.

A “computer system” is a system for electronic data processing that processes data according to programmable calculation rules. Such a system ordinarily comprises a “computer”, the unit comprising a processor for conducting logical operations, and peripherals.

In computer technology, the term “peripherals” refers to all the devices that are connected to the computer and are used for controlling the computer and/or as input or output devices. Examples thereof include monitors (screens), printers, scanners, mouse, keyboard, drives, cameras, microphones, and speakers. Internal ports and expansion cards are also considered in computer technology to constitute peripherals.

Modern computers are currently subdivided into desktop PCs, portable PCs, laptops, notebooks, netbooks and tablet PCs, and so-called hand-held devices (such as smartphones).

In some embodiments, a mobile computer system such as a smartphone, a laptop, a tablet computer or a smartwatch is used for the acquisition and/or determination and/or collection of predictor values. However, a stationary computer system (such as a desktop computer, a terminal or the like) can also be used. The computer system used for the acquisition/determination/collection of predictor values is also referred to in this description as “the first computer system”. The use of a plurality of first computer systems for the acquisition/determination/collection of predictor values is also conceivable.

In some embodiments, the first computer system is operated by the woman for whom the future bleeding pattern is to be predicted. She is also referred to here as the “user”. In this case, the term “user” refers both to use of the first computer system and to use of the computer program product according to some embodiments.

In some embodiments, the predictor values are acquired via a graphical user interface (GUI). For example, the predictor values can be entered using a (virtual) keyboard in the form of text/numerals. It is also conceivable to select predictor values from one or a plurality of virtual lists that are displayed on a screen. Entering predictor values by means of voice input using a microphone is also conceivable.

There are predictors that are associated with one or a plurality of points in time. For example, bleeding occurring in the observation period is associated with the point in time at which the bleeding occurred. The period of time of bleeding is associated with the point in time at which the bleeding occurred, and it is also associated with the point in time at which the bleeding stopped. Preferably, at least some values from at least some predictors that are associated with one or a plurality of points in time are entered in a virtual calendar via a graphical user interface. The values should preferably be entered by the user, but can also be entered by another person such as a health care provider.

In some embodiments, a virtual calendar is kept for detecting predictor values during the observation period in which the time of onset of bleeding, the period of time of bleeding, and optionally the intensity of bleeding are entered.

In some embodiments, the user operating the first computer system records the days during the observation period on which bleeding or spotting occurs and whether each occurrence is a case of bleeding or spotting.

It is also conceivable for predictor values to be queried. For example, it is conceivable that the computer program product according to some embodiments has an “alarm function” that transmits a message to the user (or another person such as a health care provider) when an event occurs and/or when a point in time is reached. For example, it is conceivable that the computer program product is configured such that a message is transferred to the user when a point in time is reached or approached at which one can expect menstrual bleeding to begin based on probability calculations and the user is queried as to whether menstrual bleeding has occurred. The user can confirm or deny the onset of menstrual bleeding by pressing a (virtual) button on the graphical user interface. She can also make indications with respect to the intensity of the bleeding.

It is further conceivable that determination of all or a portion of the predictor values is supported by one or a plurality of sensors. For example, it is conceivable for a sensor to detect the occurrence and/or stoppage of menstrual bleeding and/or the intensity thereof

It is also conceivable to use one or a plurality of sensors that are operated by the user. An example is a sensor operated by a user when she uses feminine hygiene products (such as a sanitary napkin or tampon) because of the occurrence of menstrual bleeding (an example of such a sensor is the Dash Button sold by Amazon). It is also conceivable for a sensor to be attached to or near the product that is operated by the user when she removes a product unit from a package. It is also conceivable for a sensor to be attached to a package that automatically detects when the package is opened. In this case, the signal transmitted by the sensor to the first computer system indicates that bleeding has occurred and is detected by the first computer system as data to this effect.

The sensor can be configured such that when it is operated and/or detects a signal, it transmits a message via a short-range connection (e.g. wirelessly via a transmission standard such as Bluetooth or Zigbee or the like) to a receiving device. The receiving device can be connected to a network (such as the internet) and thus transmit the message on to the units connected to the network (such as the first computer system).

The acquired predictor values are used to make predictions on the future bleeding pattern of the user. A prediction model is used for this purpose. It is also conceivable to make a prediction based on a plurality of prediction models; in order to simplify terminology, however, it is assumed in the following that there is a single prediction model. For example, the prediction model can be determined based on a study in which, as a rule, a large number of women participate in whom an intrauterine active-ingredient-releasing system has been inserted. It is conceivable that based on the data obtained in such a study, an artificial neural network or another artificial intelligence system is trained (machine learning). The trained system can then be used as a prediction model.

In some embodiments, a prediction model is created based on a random forest approach.

In some embodiments, a prediction model is prepared based on a regression tree.

The prediction of the future bleeding pattern is preferably carried out for a prediction time period. This means that based on the bleeding pattern in the observation period and optionally based on further predictors, the bleeding pattern is predicted for a prediction time period following the observation period.

In the studies on which some embodiments described herein are based, among other periods, a prediction time period of 180 days was used. Therefore, for example, if the observation period is 90 days, prediction of the bleeding pattern is carried out for a period of 91 to 270 days after insertion of the intrauterine active-ingredient-releasing system. However, other durations of the prediction time period are also conceivable.

Prediction is preferably carried out based on a classification model. The classification model assigns the woman to one of several bleeding pattern categories based on the acquired predictors. The bleeding pattern categories differ with respect to the bleeding intensity to be expected during the prediction time period. Women who are assigned to a particular bleeding pattern category show a similar bleeding pattern.

The category to which a woman is assigned thus indicates with a determinable degree of probability the type of menstrual bleeding that will occur after the observation period (during the prediction time period) (e.g. with respect to the time of onset, duration, and/or intensity). The assignment of the woman to a category of women having a certain bleeding pattern is also referred to as classification.

The number of respective categories having similar bleeding patterns is preferably two, three, four, five, six, seven, or eight.

In the case of four categories, for example, assignment of a woman to one of the following categories could be carried out:

Category I: amenorrhea

Category II: mainly spotting

Category III: regular bleeding

Category IV: irregular bleeding

The terms “bleeding” and “spotting” are preferably understood according to the definition of the World Health Organisation (WHO) (Belsey E. M. et al.: The analysis of vaginal bleeding patterns induced by fertility regulating methods, Contraception 1986, 34, 253-260):
Bleeding: vaginal blood loss that requires the use of a hygienic product such as a sanitary napkin or tampon
Spotting: vaginal blood loss that does not require the use of a hygienic product
Bleeding day: a day on which bleeding occurs
Spotting day: a day on which spotting occurs

Other category descriptions/definitions are conceivable.

A particularly preferred embodiment involves classification in three categories:

Category (20): predominant bleeding pattern: amenorrhea

Category (30): predominant bleeding pattern: spotting

Category (40): predominant bleeding pattern: bleeding

For example, the categories can be defined based on the relative frequency of bleeding or spotting days.

Table 1 shows an example definition of the above-mentioned categories (20), (30) and (40).

TABLE 1
Example definition of bleeding pattern categories

| Category | Criteria |
|---|---|
| Category (20): predominant | Fewer than 5% spotting days and |
| bleeding pattern: amenorrhea | fewer than 1% bleeding days |
| Category (30): predominant | Not belonging to category (20) and |
| bleeding pattern: spotting | fewer than 5% bleeding days |
| Category (40): predominant | Belonging neither to category (20) |
| bleeding pattern: bleeding | nor category (30) |

In some embodiments, prediction is additionally carried out with respect to the regularity of bleeding for those women for whom bleeding and/or spotting are predicted.

For example, such an indication on the regularity of bleeding can be that rather regular or rather irregular bleeding is predicted for a woman. It is also conceivable that the regularity is indicated by a value, e.g. 100% for an absolutely regular period, in which there is always exactly the same number of days between the beginning of a period and the beginning of the following period, and 0% for a period that shows no regularity (random bleeding). A colour code is also conceivable that varies for example from green (regular) via yellow (slight fluctuations of 3 to 7 days) to red (irregular, fluctuations of more than 7 days).

An indication concerning the period duration to be expected and/or the average fluctuation range of the period duration is also conceivable.

In some embodiments, an indication on the probability of regularity is made based on a logistic regression model.

The basis of the prediction on regularity is preferably a calculated period length that is determined based on the number of bleeding days in the observation period or the prediction time period using an autocorrelation function. The period length (also referred to as cycle length or cycle duration) ordinarily fluctuates between 21 and 35 days. The bleeding intensities as a function of time constitute a bleeding time function that repeats itself with absolute regularity every 21 to 35 days. If one thus shifts this bleeding time function by the amount of the period length, the shifted bleeding time function will overlap with the unshifted bleeding time function. If the time function is shifted multiple times by a specified period each time (e.g. one day) and one calculates the respective autocorrelation values between the shifted and unshifted function, a maximum autocorrelation for a shift will be obtained that corresponds to the period length. In this manner, a period length (shift value at which an autocorrelation maximum occurs) can be calculated. The parameter of the autocorrelation value can further be used as a parameter for regularity. The greater the value, the more regular the period is.

However, it is also conceivable to determine regularity based on other and/or further parameters.

In some embodiments, the bleeding time function is supplied to two time series models; one of the models has a seasonal parameter (calculated period length); the other model dispenses with such a parameter; the time series model that yields a higher information criterion (such as the Akaike information criterion) describes the bleeding time function better; if this is the model with seasonal parameters, a rather regular period is present; if it is the model without seasonal parameters, a rather irregular period is present.

In some embodiments, the prediction of the bleeding pattern is carried out based on a regression tree. FIG. 4 shows an example of a successful approach for this purpose. Women can be divided into two groups based on the number of bleeding days in the observation period: a group with more bleeding days in the prediction time period and a group with fewer bleeding days in the prediction time period. The group of women with more bleeding days can be further divided into two groups based on the autocorrelation values of the bleeding time function in the observation period: a group in which the bleeding in the prediction time period is rather irregular and a group in which the bleeding in the prediction time period is rather regular. The group of the women with fewer bleeding days can be divided into two groups based on the number of spotting days in the observation period: a group with more spotting days in the prediction time period and a group with fewer spotting days in the prediction time period.

In some embodiments, an individual future bleeding pattern for the respective woman is determined based on predictors and displayed. For this purpose, the woman is first assigned to a category based on predictors, the bleeding pattern to be expected for the category is determined, and the determined bleeding pattern is individually adapted to the woman. Such an adaptation can consist for example in adaptation of points in time of bleeding to be expected to the individual prior bleeding pattern of the woman. In such adaptation, for example, the point in time of the last bleeding and the average period duration of a woman can be used in order to predict the points in time (and optionally the intensity) of the future bleeding of the woman in a more individual manner.

The prediction of the future bleeding pattern is carried out according to some embodiments by means of a predictor unit.

The predictor unit is a unit that receives predictor values, supplies them to a prediction model (or multiple prediction models), and uses the prediction model to determine the future bleeding pattern of a user.

The predictor unit preferably receives the predictor values (or at least a portion of the predictor values) directly from the acquisition unit, carries out the prediction (e.g. a classification), and transmits the result of the prediction (e.g. the classification) to an output unit.

The predictor unit is preferably a component of a computer system. In some embodiments, the predictor unit is a component of the computer system with which the predictors values are acquired, i.e. in this case the one referred to as the “first computer system”. The first computer system can be a mobile computer system (laptop, tablet computer, smartwatch, smartphone or the like) or a stationary computer system (desktop computer, terminal or the like). It is preferably a mobile computer system.

However, the predictor unit can also be a component of a computer system that is not identical to the first computer system. Such a separate computer system that comprises a predictor unit is also referred to here as a “second computer system”. In the case of a first computer system comprising an acquisition unit and a separate second computer system comprising a predictor unit, the predictor values must be transmitted by the first computer system to the second computer system in order to allow prediction (e.g. classification) to be carried out. Such a data transmission can for example be carried out using a data carrier on which the data are stored by the first computer system and from which the data are read by the second computer system (loaded into working memory). Examples of such a data carrier are diskettes, compact disks, hard drives, USB sticks, memory cards and the like.

In some embodiments, the transmission of data (e.g. predictor values) from the first computer system to the second computer system (and optionally vice versa from the second computer system to the first computer system) is carried out via a network, preferably via the internet and/or via a cellular network.

A reason for splitting data acquisition and prediction onto two different computer systems could be that more computing power is required for the prediction of a bleeding pattern of a user than for example for determining the predictor values. In such a case, a relatively simple and thus relatively inexpensive first computer could be used for acquisition of the predictor values, such as a smartphone or a smartwatch. Use of a smartphone or a smartwatch as an acquisition unit would also have the advantage that such devices constantly accompany many users, so that in the event of unexpectedly occurring or also expected bleeding, a user can incorporate corresponding data into the smartphone or the smartwatch.

The first computer system can be configured such that, for example in the case of a minimum data amount, it transmits the data (predictor values) via the cellular network or via a WLAN network and the internet to a separate second computer system with a relatively high computing power. Evaluation of the predictor values and prediction of the bleeding pattern then take place on the second computer system. Moreover, such a system comprising separate computer systems would have the advantage of allowing the classification model to be continuously improved and updated without requiring updating of data acquisition software on the first computer system. The second computer system could also take over prediction for multiple users who are connected with their first computer systems via a network to the second (central) computer system.

It is conceivable for prediction of the bleeding pattern to take place only when “all envisaged” predictor values have been acquired. For example, it is conceivable for predictor values to be acquired using the first computer system in that predictor values are retrieved and/or predictor values are entered in fields of a graphical user interface, and the predictor values acquired in this manner are supplied for prediction only when all of the predictor values have been retrieved and entered.

However, it is also conceivable for predictor values to be continuously acquired and supplied for prediction in order to generate a constantly improved (increasingly accurate) prediction.

Procedures carried out between the two aforementioned procedures are conceivable; for example, prediction may take place only when a minimum number and/or a defined number of predictor values have been acquired; the prediction can be updated if further predictor values become available. For example, the minimum number of predictor values can be defined such that a prediction can only be made from the minimum number of predictor values having a probability above a defined threshold value (e.g. 50%).

In some embodiments, a first set of predictor values is acquired and a first prediction is made. Preferably, this first set of predictor values constitutes values that are available/known at the time of insertion of an intrauterine active-ingredient-releasing system. After the first prediction, further predictor values are acquired, preferably when they occur (in time). These further predictor values are then continuously supplied to the predictor unit or supplied to the predictor unit at defined points in time or on occurrence of defined events in order to produce a second, third and/or fourth prediction. Ordinarily, prediction improves (becomes more accurate, with less uncertainty) with an increasing number of available predictor values.

After the future bleeding pattern of a user has been determined, data on the determined future bleeding pattern are output to the user and/or another person related to the user, such as a relative, a physician and/or a caregiver.

Outputting of the data preferably takes place via a screen (e.g. as a text message and/or a graphic) and/or via speakers (e.g. as a voice message) of the first computer system. However, it is also conceivable for outputting to take place on the second computer system, for example in cases where the second computer system is operated by a physician or a similar person.

Outputting can comprise the following data
intensity of future menstrual bleeding and/or
frequency of future menstrual bleeding and/or
duration of future menstrual bleeding and/or
belonging to a defined category of women with a similar bleeding pattern and/or
regularity of future menstrual bleeding.

In some embodiments, future bleeding to be expected and the expected intensity thereof are entered in a virtual calendar so that the user can read from the calendar when bleeding will occur and how heavy it will probably be. The calendar is preferably equipped with an alarm function that warns the user of bleeding before it occurs, for example 72 and/or 60 and/or 48 and/or 36 and/or 24 and/or 12 and/or 6 and/or 2 hours before and/or at another time point.

In some embodiments, an indication is also made on the accuracy of the prediction. This applies particularly in cases in which a plurality of predictions are generated, for example because increasingly numerous predictor values become available and are determined with time. The indication concerning accuracy can take place for example in the form of indication of the probability of occurrence of a prediction. This indication can be supported with graphical means, for example in colour. For example, an indication on the increasing probability of occurrence of the prediction can be presented with an increasingly dark colour or grey tone.

The system according to some embodiments can also be configured such that the prediction on the future bleeding pattern of a woman is displayed to a physician. The term “physician” is used in this description as a generic term for people professionally engaged in human health care.

It is for example conceivable for a woman using a mobile first computer system to determine at least the bleeding pattern in an observation period after insertion of an intrauterine active-ingredient-releasing system. In a follow-up examination, the physician and/or the woman transfers the collected data to a second computer system, on which a prediction can be generated on the future bleeding pattern of the women based on the data collected, and if applicable, further data on the user, and indicated to the physician so that he/she can advise the women as to whether measures should be taken.

A computer program product is also subject matter of some embodiments. The computer program product comprises a data carrier on which a computer program is stored that can be loaded into the working memory of a computer system, where it causes the computer system to carry out one or a plurality of steps of the method according to some embodiments.

Some embodiments may comprise the situation in which functions of the computer program are distributed over a plurality of computer systems. As described above, it is therefore conceivable for the acquisition unit to be a component of a computer system, while the predictor unit is a component of another (separate) computer system. It is also conceivable for a plurality of computer systems to be used for determination and/or prediction. The output unit can also be a component of a separate computer system.

In the following, various embodiments are explained in further detail with reference to figures and preferred embodiments, without this being intended to limit the invention to the features shown in the figures or the described embodiments.

### BRIEF DESCRIPTION OF THE FIGURES

The figures are as follows:

FIG. 1 shows the result of a study of 1351 women in whom a hormone-releasing IUD marketed under the brand name Kyleena® was inserted on day 0, according to some embodiments.

FIG. 2 shows the same diagram as FIG. 1, with a prediction time period (1) and an observation period (2) included.

FIG. 3 shows an example of category formation for the bleeding data in prediction time period (1) of FIG. 2.

FIG. 4 shows a further example of category formation for the bleeding data in prediction time period (1) of FIG. 2.

FIG. 5 shows the distributions of the bleeding days of the two groups resulting from these rules. Despite overlapping, a clear difference between the two distributions can be seen.

FIG. 6 shows the distributions of the spotting days of the two groups arising in the “fewer bleeding days” group.

FIG. 7 shows the distributions of the maximum autocorrelation function values of the two groups arising in the “more bleeding days” group.

### DETAILED DESCRIPTION

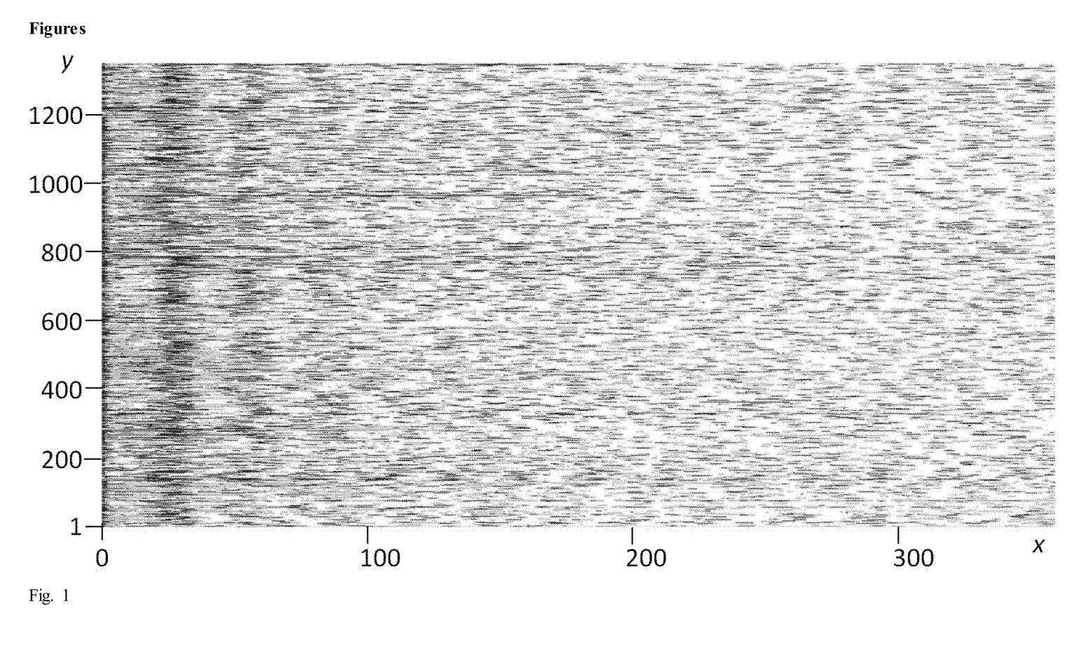

FIG. 1 shows the result of a study of 1351 women in whom a hormone-releasing IUD marketed under the brand name Kyleena® was inserted on day 0, according to some embodiments.

For the 1351 women (y axis), the figure shows on which of 360 days (x axis) after insertion of the hormone-releasing IUD bleeding occurred and how heavy this respective bleeding was (bleeding data; increasing grey value with increasing bleeding intensity).

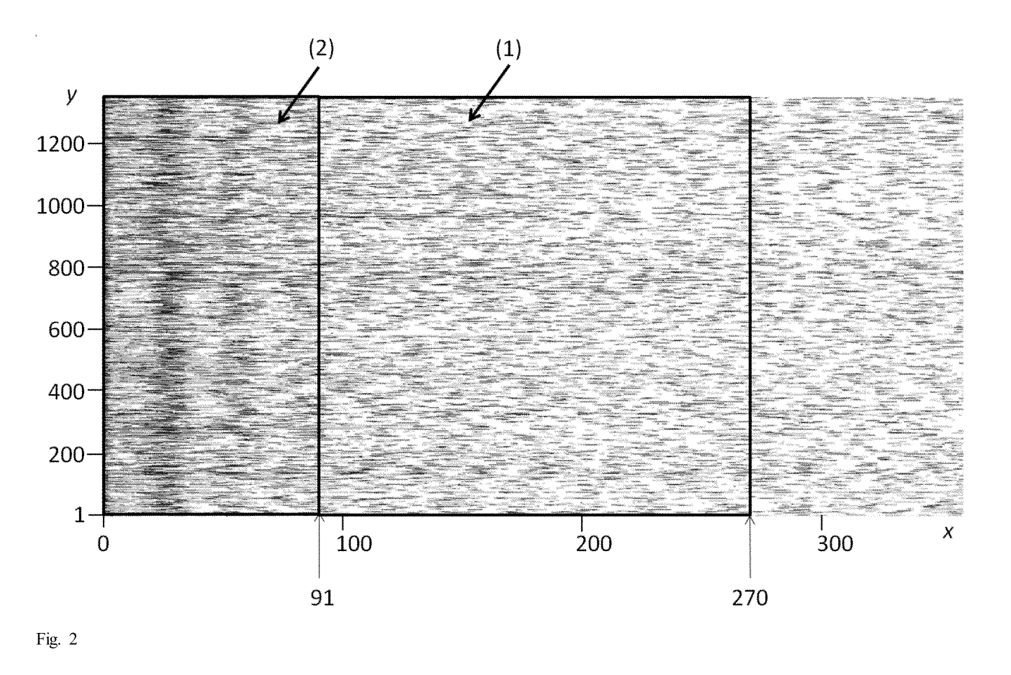

FIG. 2 shows the same diagram as FIG. 1, with a prediction time period (1) and an observation period (2) included.

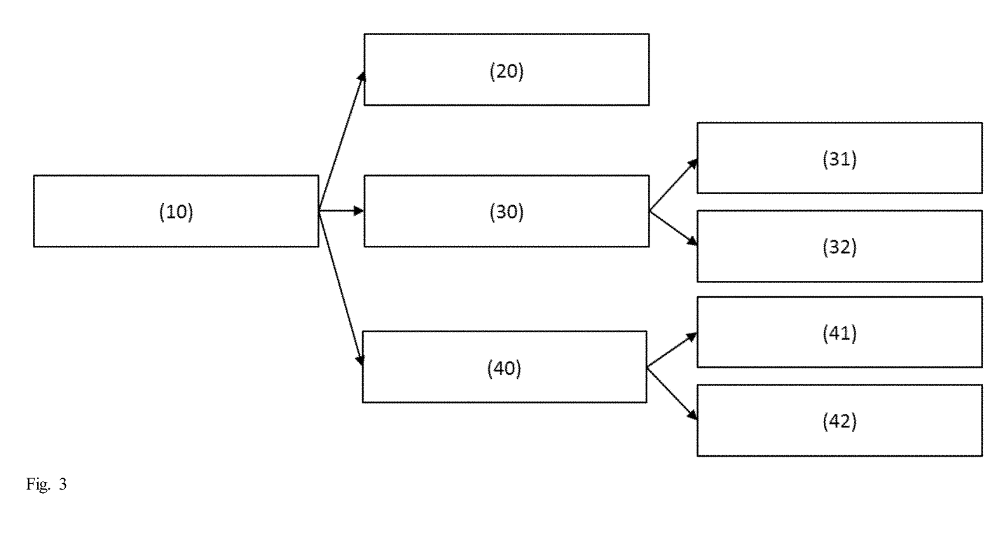

The bleeding data in the period from day 91 to day 270 were further investigated in order to identify categories with a similar bleeding pattern. This range of the bleeding data (from day 91 to day 270) is also referred to in this description as prediction time period (1). Categories were defined based on bleeding and spotting days (cf. FIG. 3, categories (20), (30), (40) and the accompanying description).

It was found that these categories can be predicted, among other factors, from the bleeding data of the observation period (2) that begins with insertion of the hormone-releasing IUD and lasts through day 90. For prediction purposes, a classification model was developed that predicts the bleeding pattern category into which a women will fall based on predictors. The classification model was developed using the random forest method.

The random forest method is a method of machine learning that can be used for example as a classification or regression method. It is based on the construction of multiple decision trees. These are also classification methods.

The object of a decision tree is to create a model that automatically predicts the class or category of a target variable based on predictor variables. A decision tree is always composed of a root node, as many inner nodes as desired, and at least two leaves. Each node represents a logical rule and each leaf an answer to the decision-making problem. If multiple predictor values are available, the variable is selected for the first node of the tree that leads to the “best” population distribution with respect to the target variables. For example, this can be calculated using criteria such as the Gini coefficient or the information gain. The tree than continues to “grow” until no further information gain can be achieved by adding further variables.

The random forest algorithm is a variation of the simple decision tree. Instead of a simple tree, many (e.g. 1000, 10,000) different decision models are calculated. For this purpose, a certain number of variables are first randomly selected for each tree from the entirety of predictor variables. A decision tree is then calculated based on this number. This process is repeated multiple times in the random forest algorithm so that a “forest” has been generated at the end of the method. If a new observation is to be classified using the model, the prediction is calculated from each individual tree, and a test is then conducted to see which category was predicted by the most trees. This then emerges from the model as the prediction result.

As there is a difference in the effects of misclassifications in the present case, a cost matrix was also integrated in order to create the model. In this case, misclassifications that are “farther away” from the true category were given twice the weight of misclassifications falling into adjacent categories.

In cases where a “new” test subject not included in the model calculations wishes to obtain data on her future bleeding pattern, the characteristics of the predictor variables and the bleeding pattern of the first 90 days are used. The test subject then receives information on which bleeding category has the highest probability of occurring.

The calculated model was calculated based on 73 predictor variables and 1351 observations. A three-fold cross validation was carried out in order to calculate error rates and confusion matrices. For further sensitivity testing of the final model, said model was tested on two further data sets. These are derived from clinical studies of intrauterine active-ingredient-releasing systems.

The accuracy of the prediction was calculated by carrying out a three-fold cross validation 1000 times. This yielded an average rate of correct classifications of 70.4%. The confusion matrix is shown below (Table 2). It should be noted that the model has been adapted such that the misclassifications occurring fall into “adjacent” clusters to the extent possible.

TABLE 2
Confusion matrix

| | | Predicted clusters | | | |
|---|---|---|---|---|---|
| | | Predominant | Predominant | Predominant | |
| | | bleeding pattern: | bleeding pattern: | bleeding pattern: | |
| | | amenorrhea | spotting | bleeding | Total |
| True | Predominant bleeding | 55 | 71 | 12 | 139 |
| clusters | pattern: amenorrhea | | | | |
| | Predominant bleeding | 22 | 324 | 132 | 478 |
| | pattern: spotting | | | | |
| | Predominant bleeding | 5 | 157 | 572 | 734 |
| | pattern: bleeding | | | | |
| | Total | 82 | 552 | 717 | 1351 |

| | | | | |
|---|---|---|---|---|
| % correct predictions* | 67.1% | 58.7% | 79.8% | 70.4% |
| % correct or better than predicted** | 67.1% | 71.5% | 100% | 86.4% |

The final model was also tested on further independent data sets not previously included in the modelling. The results showed similar rates of correct classifications of 69% and 72.2%.

Table 3 is a summary of the predictors included in developing the classification model:

TABLE 3
Predictors for predicting menstrual bleeding patterns

| Type | Description | Period |
|---|---|---|
| Baseline data | Age | |
| | Race | |
| | Country | |
| | Weight | |
| | Height | |
| | BMI | |
| | Alcohol consumption | |
| | Smoking status | |
| | Tobacco consumption | |
| | Parity | |
| | Average duration of withdrawal | |
| | bleeding | |
| | Average cycle length | |
| | Number of abortions | |
| | Number of births | |
| | Number of caesarean sections | |
| | Number of ectopic pregnancies | |
| | Number of pregnancies | |
| | Number of vaginal deliveries | |
| | Average intensity of withdrawal | |
| | bleeding | |
| | Cycle regularity | |
| | Previous contraception method | |
| | Educational level | |
| Variables derived | Number of bleeding days | Days 1-30 |
| from bleeding diary | | Days 31-60 |
| | | Days 61-90 |
| | | Days 31-90 |
| | | Days 1-90 |
| | Number of bleeding/spotting days | Days 1-30 |
| | | Days 31-60 |
| | | Days 61-90 |
| | | Days 31-90 |
| | | Days 1-90 |
| | Number of spotting days | Days 1-30 |
| | | Days 31-60 |
| | | Days 61-90 |
| | | Days 31-90 |
| | | Days 1-90 |
| | Number of bleeding episodes | Days 1-30 |
| | | Days 31-60 |
| | | Days 61-90 |
| | | Days 31-90 |
| | | Days 1-90 |
| | Number of spotting episodes | Days 1-30 |
| | | Days 31-60 |
| | | Days 61-90 |
| | | Days 31-90 |
| | | Days 1-90 |
| | Number of bleeding and spotting | Days 1-30 |
| | episodes | Days 31-60 |
| | | Days 61-90 |
| | | Days 31-90 |
| | | Days 1-90 |
| | Average duration of bleeding and | Days 1-30 |
| | spotting episodes | Days 31-60 |
| | | Days 61-90 |
| | | Days 31-90 |
| | | Days 1-90 |
| | Average duration of spotting | Days 1-30 |
| | episodes | Days 31-60 |
| | | Days 61-90 |
| | | Days 31-90 |
| | | Days 1-90 |
| | Maximum duration of bleeding and | Days 1-30 |
| | spotting episodes | Days 31-60 |
| | | Days 61-90 |
| | | Days 31-90 |
| | | Days 1-90 |
| | Maximum duration of spotting | Days 1-30 |
| | episodes | Days 31-60 |
| | | Days 61-90 |
| | | Days 31-90 |
| | | Days 1-90 |
| | Autocorrelation function maximum | Days 31-90 |
| | | Days 1-90 |

In addition to bleeding intensity (categories (20), (30), (40)), the regularity of a woman's menstrual cycle was to be classified (categories (31), (32), (41), (42)). For this purpose, the autocorrelation function for various time intervals of between 21 and 35 days was calculated for the bleeding intensity of each woman as a function of time. The autocorrelation function maximum yields the most probable cycle length of the respective woman. Based on the cycle length determined in this manner, two time series models per test subject are then calculated. One of the models comprises a seasonal component that is based on the calculated cycle length. The other model does not comprise this component. The AIC criterion is used to determine which model comprises the greater information content. If this is the model with a seasonal component, the cycle of the test subject is defined as regular. If the model without a seasonal component shows the better value, the cycle is defined as irregular.

A logistic lasso regression model was adapted based on calculation of cycle regularity for the test subjects of the analysis data set. In this case, the regularity is defined as a binary target value, and all predictor values are taken as potential influencing variables. The lasso method is used to select predictors with respect to their influence on the target values. The final logistic regression model then contains only the predictors remaining after variable selection.

There are two conceivable possibilities with respect to application of this model. For the first possibility, two different models of this type were calculated. The first model is prepared based on the data from the test subjects for whom the random forest model predicts the bleeding cluster. The second model is calculated for the test subjects for which the spotting cluster is predicted. Accordingly, the models are also used in the prediction. For a new test subject who is assigned to the bleeding cluster, the probability of a regular cycle is calculated based on the logistic regression model. For a test subject for whom primarily spotting is predicted, this probability is also calculated, but based on the logistic regression model based on the partial population of the spotting cluster.

For the second possibility of application of a logistic lasso regression model, a model was adapted with respect to the common data of the test subjects for whom the random forest model yielded classifications of bleeding or spotting. The model calculated in this manner can then be used to calculate the probability of regular bleeding for a newly-classified patient for whom the bleeding or spotting cluster is predicted.

FIG. 3 shows an example of category formation for the bleeding data in prediction time period (1) of FIG. 2.

Category (10) comprises all 1351 women in the study. The subjects are subdivided into categories (20), (30) and (40). Category (20) comprises the women showing fewer than 5% spotting days and fewer than 1% bleeding days in the prediction time period. Women in this category predominantly show amenorrhea. 139 women were assigned to category (20). Category (30) comprises the women showing fewer than 5% bleeding days in the prediction time period but not assigned to category (20). Women in this category predominantly show the bleeding pattern of spotting. 478 women were assigned to category (30). Category (40) comprises the women assigned neither to category (20) nor to category (30). Women in this primarily show the bleeding pattern of “true” bleeding. 734 women were assigned to category (40).

Categories (30) and (40) are further subdivided with respect to the regularity of spotting/bleeding. Category (31) comprises women who show regular spotting. Category (32) comprises women who show irregular spotting. Category (41) comprises women who show regular bleeding. Category (42) comprises women who show irregular bleeding.

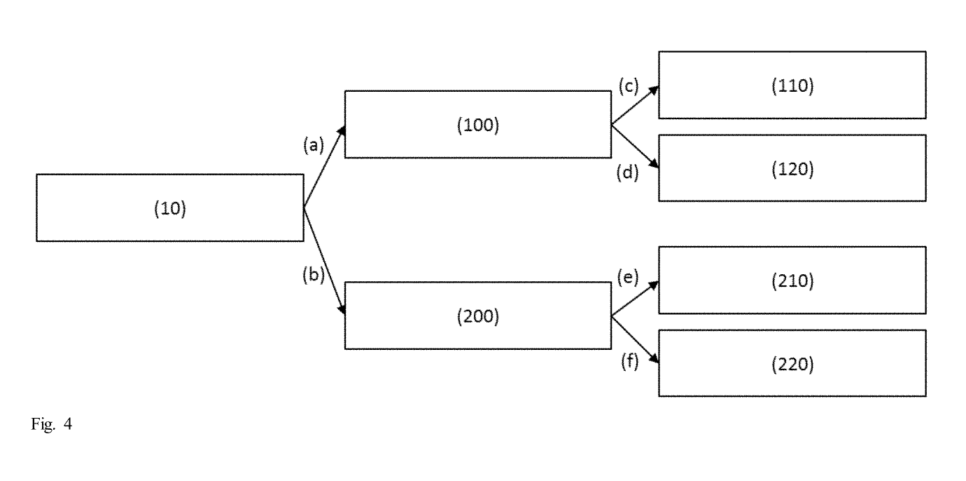

FIG. 4 shows a further example of category formation for the bleeding data in prediction time period (1) of FIG. 2.

This category formation is based on an approach different from that of the category formation according to FIG. 3; in this case, category formation is based on a regression tree. Here, the procedure generally involves subdividing a population with respect to a certain target variable into groups that differ as widely as possible. A variable is selected for subdivision purposes that is part of the predictor. In this case, the variable is selected that results in the greatest difference between the groups with respect to the target variables. The difference between the two groups is calculated and maximized based on a measure of node purity: the residual sum of squares. For this purpose, an F test criterion is used whose test statistic is the quotient of the residual sums of squares multiplied by 1/(n−1). This method is then used multiple times in order to subdivide the population into multiple groups with respect to different target variables of the prediction time period. Of interest here are the number of bleeding days, the number of spotting days, and the regularity, which is indicated by the maximum value of the autocorrelation function.

In a first step, within the first 90 days after insertion of the hormone-releasing IUD, predictors were sought that could subdivide the group of 1351 women (category (10)) into two groups differing as widely as possible with respect to the number of bleeding days in the prediction time period (categories (100) and (200)). In a second step, the women with more bleeding days (category (100)) were divided into “irregular” (category (110)) and “regular” (category (120)) groups; the group of women with fewer bleeding days (category (200)) was divided with respect to the number of spotting days into a category with more spotting (category (210)) and a category with less spotting (category (220)). 495 women were assigned to category (100). 856 women were assigned to category (200). 243 women were assigned to category (110). 251 women were assigned to category (120). 369 women were assigned to category (210). 487 women were assigned to category (220).

Table 4 shows a summary of the criteria for classification of the groups.

TABLE 4
Criteria for the classification of categories in FIG. 4.

| | |
|---|---|
| (a) | Fewer than 20 days with bleeding or spotting in the first |
| | 90 days after insertion of the hormone-releasing IUD |
| (b) | More than 19 bleeding days in the first 90 days after |
| | insertion of the hormone-releasing IUD |
| (c) | Value of the autocorrelation function greater than |
| | or equal to 0.3 |
| (d) | Value of the autocorrelation function less than 0.3 |
| (e) | More than 4 spotting days in the first 90 days after |
| | insertion of the hormone-releasing IUD |
| (f) | Fewer than 5 spotting days in the first 90 days after |
| | insertion of the hormone-releasing IUD |

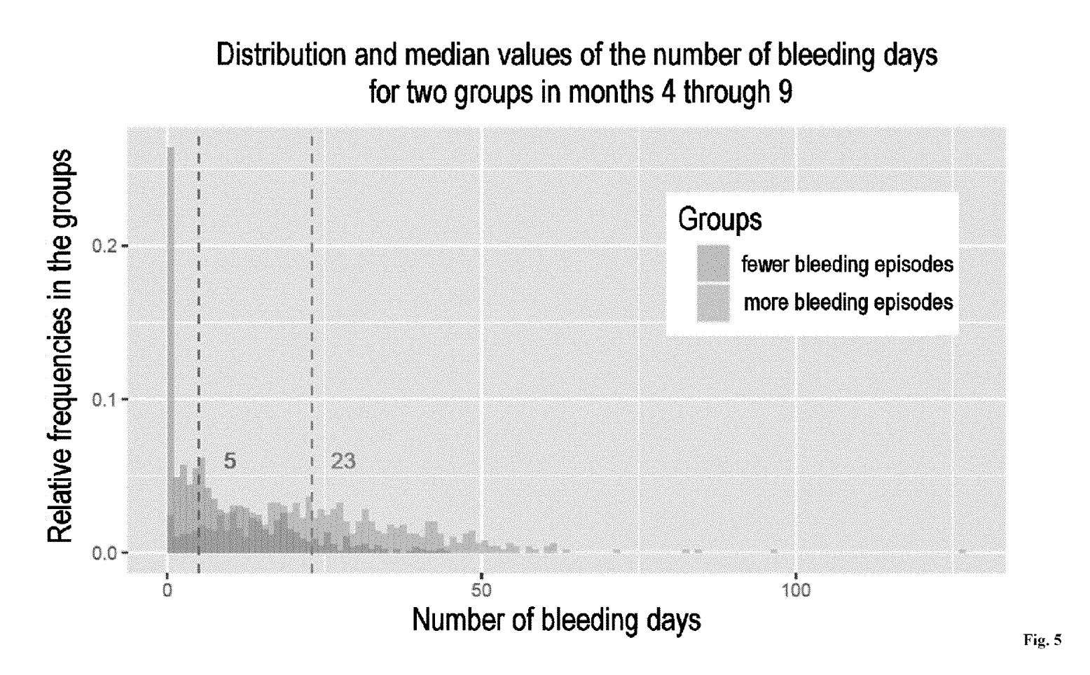

FIG. 5 shows the distributions of the bleeding days of the two groups resulting from these rules. Despite overlapping, a clear difference between the two distributions can be seen.

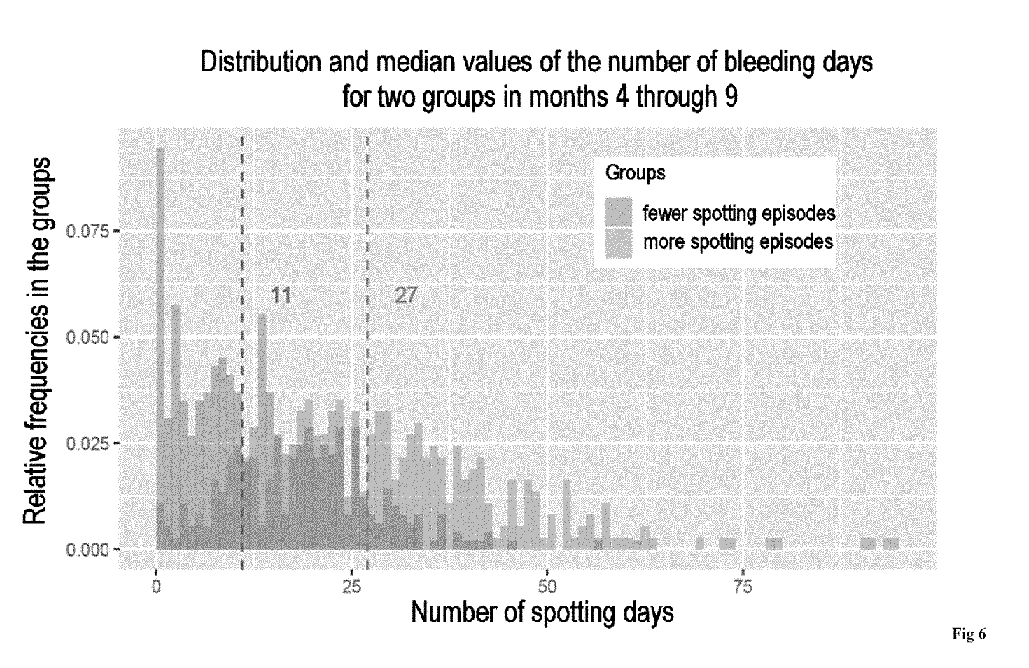

FIG. 6 shows the distributions of the spotting days of the two groups arising in the “fewer bleeding days” group.

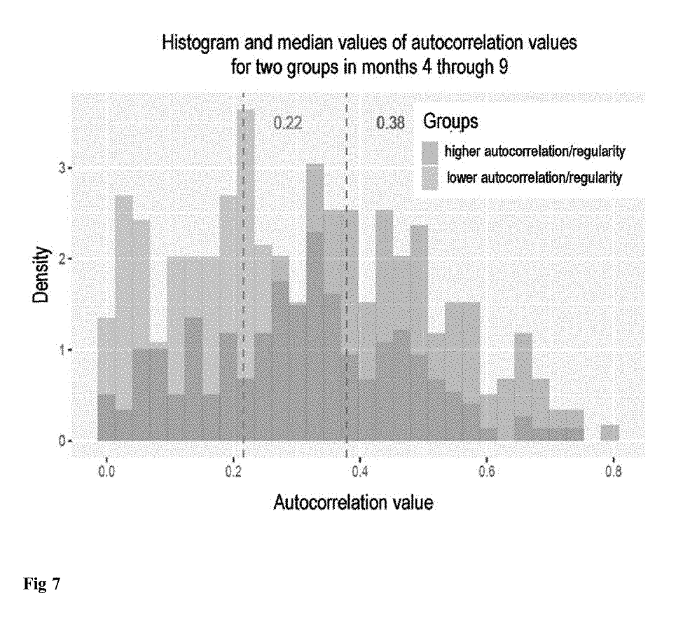

FIG. 7 shows the distributions of the maximum autocorrelation function values of the two groups arising in the “more bleeding days” group.

In the following, various embodiments of the method according to the invention are described.

In some embodiments, a hormone-releasing IUD that releases progestin is inserted in the user. The woman downloads from an app store a computer program product according to some embodiments in the form of an app onto her smartphone, installs the computer program and starts it.

In some embodiments, the computer program is configured such that it greets the woman and asks her to enter several indications, such as her age and the time of insertion of the hormone-releasing IUD, in corresponding fields that constitute a graphical user interface or to select them from a list and enter them in a virtual calendar. The woman is now asked to indicate for a specified observation period whether she has/had bleeding, spotting, or no bleeding on the individual days of the observation period. After the observation period passes and the data have been entered, the smartphone transmits the acquired data via the cellular network and/or the internet to a second computer system. A prediction model is installed on the second computer system that receives predictor values and makes a prediction on the future bleeding pattern of the woman.

The result of the prediction is transferred from the second computer system to the first computer system and displayed for the woman on the display of the smartphone.

In some embodiments, the prediction is constantly improved based on newly-entered data. The computer program is configured such that it transmits newly-entered data (predictor values) to the second computer system from time to time. The second computer system updates the prediction using all of the previously acquired predictor values and transmits the result of the prediction to the first computer system.

In some embodiments, the computer program on the first computer system is configured such that it enters the predicted days on which bleeding is to occur in the virtual calendar. The intensity of the predicted bleeding is also entered in the calendar using a coloured encoding system. It is also conceivable for uncertainty as to the onset of bleeding on a day to be indicated in that the possibility of onset of bleeding is indicated in the calendar one or more days before and/or after the predicted day.

In some embodiments, acquisition of predictors and calculation of the prediction are carried out on a single computer system operated by a single user in whom an intrauterine active-ingredient-releasing system has been inserted. The user continuously enters predictor values in the computer system. She is guided in this process by a graphical user interface and/or a chat bot. The user can initiate calculation of a prediction by pressing a virtual button. In this case, the acquired predictor values are supplied to a prediction model that is stored on the computer system. The prediction model calculates a future bleeding pattern and displays it to the user.

In some embodiments, a woman in whom an intrauterine active-ingredient-releasing system has been inserted by a physician is asked by the physician to observe her bleeding pattern during an observation period and to acquire data on the bleeding pattern, for example using a computer system or forms she is to fill out. At the time of a follow-up examination, the physician transfers the data collected from the woman on her bleeding pattern to a computer system, and the computer system determines her future bleeding pattern based on these data and preferably based on further data on the woman as well. The result is displayed to the physician, and he can then discuss it with the woman and either print out the result of the prediction for her or transmit it to her as a file using a computer system.

1: A system for predicting the menstrual bleeding pattern of a woman using an intrauterine active-ingredient-releasing system, comprising
an acquisition unit for acquiring predictor values, wherein the acquisition unit is configured to acquire data on the bleeding pattern of a woman in an observation period, wherein the observation period begins with insertion of an intrauterine active-ingredient-releasing system and lasts at least 60 days;
a prediction unit configured to predict the future bleeding pattern of the woman based on redictor values acquired by the acquisition unit, wherein the predictor unit is based on a classification model configured to classify the woman in one of several bleeding pattern categories, wherein the several bleeding pattern categories differ with respect to bleeding intensities to be expected in a prediction time period, and wherein the prediction time period is subsequent to the observation period; and
an output unit configured to output the predicted future bleeding pattern to one or more of the woman or a physician.

2: The system of claim 1, wherein the acquisition unit is a component of a mobile computer system.

3: The system of claim 1, further comprising a first computer system for acquiring the predictor values and a second computer system for predicting the future bleeding pattern of the woman, wherein the first computer system and the second computer system are configured such that the first computer system transmits predictor values to the second computer system and the second computer system transmits the predicted future bleeding pattern to the first computer system.

4: The system of claim 1, wherein the acquisition unit, the predictor unit, and the output unit are components of a single computer system.

5: The system of claim 1, further comprising one or a plurality of sensors configured to transmit one or a plurality of signals to the acquisition unit, and wherein the acquisition unit is configured to interpret the one or a plurality of signals as one or a plurality of predictor values.

6: A method for predicting the menstrual bleeding pattern of a woman using an intrauterine active-ingredient-releasing system, comprising the following steps:
acquiring data on the bleeding pattern of a woman in an observation period, wherein the observation period begins with insertion of an intrauterine active-ingredient-releasing system and lasts at least 60 days, and acquiring one or a plurality of predictor values;
predicting a future bleeding pattern of the woman based on the predictor values using a classification model, wherein the classification model is configured to classify the woman in one of several bleeding pattern categories, wherein the bleeding pattern categories differ with respect to bleeding intensities to be expected in a prediction time period, and wherein the prediction time period is subsequent to the observation period; and
outputting the predicted future bleeding pattern to one or more of the woman or a physician.

7: The method of claim 6, wherein the observation period is from 61 days to 120 days.

8: The method of claim 6, wherein the classification model is based on a random forest approach and is configured to classify the woman to one of the following three categories: amenorrhea, spotting, and bleeding.

9: The method of claim 6, further comprising determining a future bleeding intensity pattern based on a calculated period length of the woman based on an autocorrelation of bleeding intensity data as a function of time.

10: The method of claim 6, wherein the classification model is based on a regression tree and is configured to classify the woman to one of the four following categories:
irregular bleeding, regular bleeding, more spotting, and less spotting.

11: The method of claim 6, wherein a first prediction on the future bleeding pattern is determined based on a first set of predictor values and is updated after acquisition of a second set of predictor values to generate a second prediction on the future bleeding pattern.

12: The method of claim 6, wherein predictor values for one or a plurality of the following predictors are acquired within the observation period:
points in time at which bleeding occurred,
periods of time during which bleeding continued,
intensity of bleeding
total number of bleeding days
total number of spotting days
regularity of bleeding (with respect to duration and intensity)
number of bleeding or spotting episodes.

13: The method of claim 6, wherein predictor values on one or a plurality of the following predictors are determined:
the age of the woman,
the contraception method the woman used prior to use of the intrauterine active-ingredient-releasing system,
the number of previous pregnancies/births of the woman,
the consumption of tablets and/or additive drugs,
indications on the bleeding pattern in a defined time period prior to insertion of an intrauterine active-ingredient-releasing system.

14: The method of claim 6, wherein the future bleeding pattern is entered in a virtual calendar.

15: A computer program product comprising a data carrier on which a computer program is stored that can be loaded into the working memory of one or a plurality of computer systems, wherein the computer program product causes the one or a plurality of computer systems to carry out the following steps:
acquire data on the bleeding pattern of a woman using an intrauterine active-ingredient-releasing system in an observation period, wherein the observation period begins with insertion of the intrauterine active-ingredient-releasing system and lasts at least 60 days, and acquire one or a plurality of predictor values;
predict the future bleeding pattern of the woman based on the predictor values by using a classification model, wherein the classification model is configured to classify the woman in one of several bleeding pattern categories, wherein the bleeding pattern categories differ with respect to bleeding intensities to be expected in a prediction time period, and wherein the prediction time period is subsequent to the observation period; and
output the predicted future bleeding pattern to one or more of the woman or a physician.
